(19) United States
(12) Patent Application Publication
(10) Pub. No.: US 2019/0180239 A1
(43) Pub. Date: Jun. 13, 2019

(54) SYSTEM AND METHOD FOR TREATING A PATIENT BY A PROVIDER AND RECEIVING PAYMENT FOR THE TREATMENT

(71) Applicant: Creative Venture Solutions, Ltd., Canton, OH (US)

(72) Inventors: Brandon Fabris, Massillon, OH (US); Daniel Joseph Beers, II, Massillon, OH (US)

(21) Appl. No.: 16/168,945

(22) Filed: Oct. 24, 2018

Related U.S. Application Data
(60) Provisional application No. 62/598,101, filed on Dec. 13, 2017.
(60) Provisional application No. 62/685,357, filed on Jun. 15, 2018.

(51) Int. Cl. G06Q 10/10; G16H 10/60; G06Q 40/08

(52) U.S. Cl. CPC G06Q 10/10; G06Q 40/02; G06Q 40/08; G16H 10/60

(57) ABSTRACT

A system and method is provided that effectuates member-to-member sharing of funds between bank accounts maintained at a bank. The sharing occurs subsequent to one of the members of the organization receiving a rendered service from a provider. This results in a specific sharing need that the organization is aware of. The organization directs the bank to transfer funds from one member to the member in need of paying the expense associated with the rendered service. The money from the other members that is deposited into the receiving member's account is then distributed to the provider as a payment for the rendered service. The money may be distributed directly from the bank, or the money may be provided to the organization that directs the payment to the provider.

### CROSS REFERENCE TO RELATED APPLICATIONS

This application claims the benefit of U.S. Provisional Application Ser. No. 62,598,101, filed on Dec. 13, 2017 and U.S. Provisional Application Ser. No. 62/685,357, filed on Jun. 15, 2018; the disclosures of each of which are incorporated herein by reference.

### BACKGROUND

### Technical Field

The present disclosure relates to cost sharing associations composed of members that share costs to pay for expenses of other members. Particularly, aspects of the present disclosure may relate generally to systems, devices, and methods for performing data processing operations for sharing one or more expenses, in which there is some or a significant change in the data or for performing calculation operations wherein the apparatus or method is uniquely designed for or utilized in the practice, administration, or management of an enterprise, or in the processing of financial data, and provides an apparatus or system, and corresponding methods for performing data processing or calculating operations in which a charge for services rendered is determined and shared between members of an organization. More particularly, one aspect of the present disclosure may relate to subject matter drawn to a computerized arrangement for including a mechanism for effecting a transaction and determining the amount of a service which may also be a terminal in a system, or having interface for record bearing medium or carrier for electronic funds transfer or payment credit and the associated payment effecting data bearing record or carrier. Specifically, the present disclosure relates to sharing costs directly between members of a common organization.

### Background Information

Grouping of individuals is common to achieve certain benefits or advantages. The grouping is done largely in the insurance industry. By pooling resources into a common fund, the group members are able to afford insurance coverage for such things as health, automobile and life. Without being able to pool resources, the individual would have to bear the entire costs for such things as health or automobile expenses.

With respect to health care, an average consumer may not be able to afford much more than the basic healthcare coverage if grouping of resources was not available. Cutting edge surgical or drug treatments would be difficult, if not, impossible for the average consumer based upon current medical rates being charged in the medical industry.

By placing all individuals into one group, insurance coverage and the benefits obtained thereby are made more affordable. This coverage is primarily based, though, upon the overall health of the group. There will be individuals in the group that would just use the insurance for yearly checkups and there will other individuals, who will use the system much more extensively. It is the later group that has a greater effect on the premiums in the group. For those that do not use the insurance that frequently, being lumped with the more extensive users is a downside or disadvantage. On the other side, the individuals, who demand a greater share of the benefits, have the advantage of keeping their premiums lower than usual because of the individuals that demand little if any benefits from the program.

As can be seen, one of the benefits of insurance programs is the ability to pool resources so that the group as a whole is able to share in its benefits. However, a downside of this is that the group is generally composed of individuals that either makes frequent requests for the benefits or others that do not require benefits that often. Another disadvantage of the insurance system is that the beneficiaries, at any point, could lead unhealthy or at risk lifestyles such as high risk diets, low exercise, smoking, excessive alcohol intake and the use of illicit drugs. By engaging in such lifestyles, these individuals are at a greater likelihood to use the health insurance and its benefits. With more of these people using the benefits, the more likely the premiums and associated costs with providing these benefits for all the individuals increases.

An additional disadvantage of the present insurance system for pooling resources is that the benefits are distributed to the individuals such that no other individual in the participating in the program has any real sense of what types of requests are being made and what types of service are being paid for by the insurance company. This disadvantage with such a system is that a beneficiary is less likely to be held accountable in a system where it is known how much everyone in the group has contributed and how each and every person in the group has requested in the form of benefits.

Over the centuries, friends, neighbors and communities have shared their wealth and income to meet the needs of those less fortunate or those facing calamity. Sharing in the burdens of one's neighbor might be found within the confines of the organized church and is typically a random “as needed” event. More recently, a number of organizations such as faith-based groups have begun to create associations for the specific purpose of sharing in each other's everyday needs and burdens. These organized groups are often referred to as mutual sharing associations or MSAs. MSAs are a fast growing and legitimate alternative to traditional insurance. It is estimated that more than 500,000 families will participate in associations that share in the everyday healthcare expenses of their members. As this phenomenon grows, mutual sharing is beginning to extend beyond healthcare needs and into new categories such as income protection (disability), legal protection (liability) and property protection (property and casualty).

There are many reasons for participating in a mutual sharing association. MSAs provide a sense of community for people of like values, lifestyles and faiths. MSAs typically provide visibility into the individual needs that are being shared so members can exchange prayers, words of encouragement and emotional support. MSAs can build stronger risk pools than traditional insurance by stipulating specific lifestyle requirements as a condition for membership. MSAs are traditionally “non-profit” entities and often reduce the household expenditures typically paid to “for-profit” insurance companies by more than 50%.

The future and potential impact of MSAs have yet to be felt in the marketplace. In fact, in a world where time, distance, and space is reduced by electronic networks, people of like minds, values and faiths will find it easier to meet and participate in organize exchanges of interests. Additionally, legislative changes that ease restrictions on the creation of association benefit plans will combine with the heavy promotion and marketing of health savings accounts (HSA) and high deductible “catastrophic” health care plans to generate market tailwinds and risk mitigation tools necessary for dramatic growth. MSAs, with a history and track record of accountability and stewardship, will find themselves to be at the center of an ideal market opportunity.

While the market opportunity is great, the business processes of MSAs need to be able to meet the demands of the larger general market. However, it is important to note that some of the business processes can cause distress and conflict with regulatory agencies that ultimately suppress an MSA's ability to aggressively grow its market influence.

To capitalize on their market potential and shed their regulatory burdens, MSAs need some innovative processes in their structure that accomplish the following: 1) Markets, transacts and fulfills services through an obvious context of shared values, faith, interests and community; 2) Embraces and adopts innovative uses of traditional insurance to mitigate association and member risk; 3) Creates an obvious delineation between traditional insurance and sharing by focusing sharing plans on deductibles and out-of-pocket expenses that accompany high deductible insurance plans; 4) Matches and presents specific “member need” with every “share request” in a real-time transaction initiated by the member; 5) Transacts a direct “account-to-account” process; 6) Displays all regulatory disclosures prior to the exchange (transaction) of shares; and 7) Provides complete visibility and history into all share transactions.

Thus, MSAs wanting to gain a greater influence into their respective constituent communities should begin to prepare themselves for this emerging market boom by leveraging technology to web-enable their current paper-based processes into a Virtual Share Exchange (VSE) and free themselves from the weight and suppression of regulatory concerns.

Conventional MSA sharing methods provide for the sharing of resources among a group of individuals in a processor based system in order to create a stronger risk pool includes providing data relating to a first member of the group, providing data relating to a second member of the group, receiving a request for a benefit from the first member and assigning a portion of the request to a second member.

MSA sharing methods allow for posting, publishing and transacting the sharing of eligible needs in a way that consistently meets the strict requirements of regulatory agencies. State regulatory agencies such as the Department of Insurance are keenly sensitive to how the sharing of needs and burdens is actually processed. In order to help alleviate the regulatory burdens, it is important that the business practices of MSAs create an obvious delineation from the practices of insurance companies and their indemnity contracts. While there are subtle differences between the states and their specific requirements for mutual sharing associations, there are four requirements that appear to be consistent across all states and should be complied in order to transact an exchange of mutual sharing in an effective manner. The requirements are: 1. Matching a specific share with a specific need; 2. The exchange must use a members-to-member direct sharing process and not pool funds; 3. The exchange must not build reserves; and 4. The exchange must make obvious attempts to disclose to its members that the service is not insurance and does not imply a guarantee of benefits.

### SUMMARY

Issues continue to exist with conventional insurance models that pool large amounts of funds from the members into a common fund that is used to pay claims made to the insurance company. Particularly, the conventional insurance model presents technical problems of regulatory compliance and government regulations, risks, and fees. The present disclosure provides a technical solution to overcome the technical problems associated with the conventional insurance model by providing a system that commands and effectuates the sharing of expenses for members of an organization through the use of funds that are maintained in individual and independent bank account such that member funds are not pooled prior to paying a provider.

In accordance with one aspect, an exemplary embodiment of the present disclosure may provide a system for member-to-member cost sharing comprising at least one non-transitory computer readable storage medium having instructions encoded thereon that, when executed by at least one processor, implement operations to effectuate a cost sharing function directly between members without any pooling of funds. The system effectuates a method for cost sharing directly between members comprising: effectuating a direct transfer from a first member bank account to a second member bank account in response to receiving a notification that a cost needs to be funded.

In accordance with another aspect, an exemplary embodiment of the present disclosure may provide a method of sharing costs between multiple parties including: maintaining, by a bank, a plurality of individual bank accounts that remain segregated and independent from other bank accounts; receiving funds, deposited by one of a mutual sharing association (MSA) application and a first member of the MSA, into a first bank account of the first member that is maintained by the bank, wherein the first member and the MSA application are different entities, and wherein the funds travel along a first pathway from the MSA application to the first bank account; receiving a notification that a provider has rendered a service to the first member; receiving data, in a processor, of a payment to be paid in exchange for the service that was previously provided by the provider and received by the first member; transferring funds directly from a second bank account of a second member that is maintained by the bank along a second pathway in response to a provider payment schedule provided to the bank by the application, wherein the second pathway is independent from the first pathway; and paying the provider with funds from the second member. This exemplary embodiment or another embodiment may further provide wherein paying the provider is accomplished by funds from the second member held in the second bank account moving solely along the second pathway and not along the first pathway. This exemplary embodiment or another embodiment may further provide wherein paying the provider is accomplished by funds from the second member transferred along the second pathway into the first bank account, and the funds from the second member are automatically withdrawn by the bank from the first bank account. This exemplary embodiment or another embodiment may further provide receiving, in the processor, monthly invoices generated by the MSA application containing data associated with services performed by the provider to be paid by the bank for other members of the MSA. This exemplary embodiment or another embodiment may further provide wherein receiving the monthly invoices is accomplished by a transferring from a file generated by the MSA application; wherein each file generated by the MSA application is associated with movement of funds between bank accounts along respective independent pathways such that funds are not pooled. This exemplary embodiment or another embodiment may further provide receiving data associated with a method of payment for the bank to pay the provider with funds of the second member from the first bank account of the first member, wherein the second pathway extends from the second bank account to the first bank account; and moving funds along the second pathway into the first bank account, wherein only funds from the second member move along the second pathway such that funds from the second member remains independent and segregated from other funds traveling along other pathways that terminate at the first bank account. This exemplary embodiment or another embodiment may further provide receiving updated invoices from an accounts receivable file in the MSA application, wherein the invoices travel along a common pathway from the MSA application to the bank. This exemplary embodiment or another embodiment may further provide receiving, at the bank, a payment method file from an accounts receivable at the MSA application, wherein the payment method file travels along the common pathway. This exemplary embodiment or another embodiment may further provide debiting a credit card payment of the second member in response to the received data of the payment to be paid in exchange for the service that was previously provided by the provider and received by the first member, wherein funds debited from the credit card of the second member are deposited directly into the second bank account and remain independent from funds of other members. This exemplary embodiment or another embodiment may further provide debiting an ACH payment of the second member in response to the received data of the payment to be paid in exchange for the previously provided service from the provider received by the first member, wherein funds debited from the ACH payment of the second member are deposited directly into the second bank account and remain independent from funds of other members. This exemplary embodiment or another embodiment may further provide crediting the first bank account of the first member from a transaction of the second member in response to the received data of the payment to be paid in exchange for the previously provided service from the provider received by the first member. This exemplary embodiment or another embodiment may further provide paying the provider, directly from the bank, with funds credited to the first member from the transaction of the second member. This exemplary embodiment or another embodiment may further provide paying the provider, directly from the bank, with the monies of the second member in response to the received data of the payment to be paid in exchange for the previously provided service from the provider received by the first member, wherein the funds from the second member that move along the second pathway remain independent from other pathways along which funds from other members move. This exemplary embodiment or another embodiment may further provide receiving, at the bank, invoices generated by the MSA application in response to the first member submitting a bill to the MSA application; and identifying the bill to be paid for services provided by the provider to the first member, and initiating a payment for the bill to be sent directly from one of the second bank account and the first bank account containing funds sourced from the second member. This exemplary embodiment or another embodiment may further provide receiving, at the bank, invoices generated by the MSA application in response to the provider submitting a bill to the MSA application for services rendered to the first member; and identifying the bill to be paid for services provided by the provider to the first member, and initiating a payment for the bill. This exemplary embodiment or another embodiment may further provide paying the provider, directly from the bank, with monies from the second member moving along the second pathways for the services provided to the first member in response to receiving, at the bank, data associated with invoices supplied by the provider. This exemplary embodiment or another embodiment may further provide activating a payment of a bill from other member bank accounts that are maintained independent such that no funds are pooled in a common account. This exemplary embodiment or another embodiment may further provide accumulating payment orders from the MSA application over a period of time and maintaining the payment orders in a period of time, and paying a provider directly with mimicking a visual representation of the application. This exemplary embodiment or another embodiment may further provide mimicking the MSA application when paying the provider such that the payment appears, as observed by the provider, to be coming from the MSA application, when the payment is actually coming from the bank. This exemplary embodiment or another embodiment may further provide aggregating payment orders from the MSA application so as to enable the bank to batch schedule payments to either the MSA application or the provider.

In accordance with another aspect, an exemplary embodiment of the present disclosure may provide A method for treating a patient by a medical provider and receiving payment for the treatment comprising: treating, by a provider, a first patient who is a member of a mutual cost sharing association or organization (MSA) that does not collectively pool monies of MSA members that are provided to the MSA, wherein treating the first patient with a medical service defines a rendered service by the provider; identifying, by the provider, the rendered service in a digital format; transmitting the digital format of the rendered service from a provider computer to the MSA; and receiving, by the provider, a payment from a bank in operative communication with the MSA for the rendered service to the first patient, wherein the payment includes monies from other MSA members having individual bank accounts at the bank. This exemplary embodiment or another embodiment may further provide wherein the payment from the bank to the provider does not include money from the first patient. This exemplary embodiment or another embodiment may further provide terminating, by the provider, an accounts receivable balance in response to receiving payment from the bank that includes money from other MSA members and not the first patient. This exemplary embodiment or another embodiment may further provide accessing, by the provider, a registry of at least one payment made by the bank; and confirming, by the provider, the at least one payment was paid in full for the rendered service to the first patient. This exemplary embodiment or another embodiment may further provide agreeing, by the provider, to provider a second rendered service to the first patient in response to receiving payment from the bank, wherein the second render service is to be paid with monies from other MSA members that are not pooled prior to paying the provider. This exemplary embodiment or another embodiment may further provide processing, by the computer under the control of the provider, the payment defined by a plurality of monies form other MSA members distinct from the first patient. This exemplary embodiment or another embodiment may further provide aggregating, by the provider in a provider bank account, a plurality of monies received from other MSA members for the rendered service to the first patient, wherein the aggregating by the provider occurs after depositing the plurality of monies in the provider bank account such that no pooling of money occurs at the MSA or at the bank. This exemplary embodiment or another embodiment may further provide alerting, by the provider, the MSA in response to a notification from a provider bank that an insufficient amount of money was provided to the provider from the bank for the rendered service to the first patient. This exemplary embodiment or another embodiment may further provide associating, by the provider, the payment of monies from other MSA members to an account receivable of the first patient; and confirming to the MSA that the account receivable of the first patient has been paid by the bank without the MSA pooling money of the MSA members. This exemplary embodiment or another embodiment may further provide allotting, by the provider, a number of line item entries at least equal to a number of payments received by the bank representing monies that are not pooled and retained in separate and independent bank accounts of other MSA members. This exemplary embodiment or another embodiment may further provide authenticating, by the provider, that the payment received from the bank has been paid from with monies from other MSA members that are not pooled prior to paying the provider. This exemplary embodiment or another embodiment may further provide basing additional services rendered by the provider to the first member on payment of the rendered service with monies from other MSA members that are not pooled prior to paying the provider. This exemplary embodiment or another embodiment may further provide billing, by the provider, the MSA to establish an account receivable at the provider and thereby establishing an expense for the first member at the MSA; and notifying, by the provider, the MSA that the payment has been received with monies from other MSA members that are not pooled prior to paying the provider so that the MSA notes that the expense for the first member has been paid. This exemplary embodiment or another embodiment may further provide bypassing the bank, by the provider, to notify the MSA that the payment has been received for the rendered service to the first member. This exemplary embodiment or another embodiment may further provide combining, by the provider, at a provider bank account monies received from the bank as payment for the rendered service, wherein monies are pooled by the provider at the provider bank account and not before. This exemplary embodiment or another embodiment may further provide decreasing, by the provider, an account receivable entry associated with the rendered service to the first member in response to receiving payment from the bank for the rendered service with monies from other MSA members that are not pooled prior to paying the provider. This exemplary embodiment or another embodiment may further provide diagnosing, by the provider, the first member with a diagnosis as the rendered service prior to receiving payment from the bank for the rendered service with monies from other MSA members that are not pooled prior to paying the provider. This exemplary embodiment or another embodiment may further provide directing, by the provider, a provider bank account to receive receiving payment from the bank for the rendered service with monies from other MSA members that are not pooled prior to paying the provider is response to the MSA notifying the bank that a payment is to be made directly from the bank to the provider. This exemplary embodiment or another embodiment may further provide grouping, by the provider, at a provider bank account monies from other MSA members as the payment for the rendered service. This exemplary embodiment or another embodiment may further provide generating, by the provider, a notification sent to the MSA with instructions to be provided to the bank for the payment to be sent to a provider bank account. This exemplary embodiment or another embodiment may further provide instructing, by the provider, the MSA to initiate the payment from the bank with monies from other MSA members that are not pooled prior to paying the provider, wherein instructing the MSA is accomplished by sending an invoice for the rendered service. This exemplary embodiment or another embodiment may further provide initiating, by the provider, a second rendered service to the first member in response to the bank paying the provider the payment with monies from other MSA members. This exemplary embodiment or another embodiment may further provide limiting, by the provider, an additional number of services that can be rendered to the first member until the provider receives payment from the bank for the rendered service with monies from other MSA members that are not pooled prior to paying the provider. This exemplary embodiment or another embodiment may further provide maintaining, by the provider, a provider bank account; and receiving in the provider bank account monies from other MSA members that are not pooled prior to paying the provider and are pooled by the bank, not the MSA, at the time the provider is paid. This exemplary embodiment or another embodiment may further provide minimizing, by the provider, additional services to be rendered to the first member until the provider has received payment from the bank for the rendered service with monies from other MSA members that are not pooled prior to paying the provider and are pooled by the bank, not the MSA, at the time the provider is paid. This exemplary embodiment or another embodiment may further provide negotiating, by the provider, with the MSA a cost associated with the service, prior to rendering the service to the first member, wherein the cost associated with the service is based, at least partially, on a guarantee by the MSA that the bank will pay for the rendered service with monies from other MSA members that are not pooled prior to paying the provider and are pooled by the bank, not the MSA, at the time the provider is paid. This exemplary embodiment or another embodiment may further provide participating, by the provider, with the MSA to cooperate with the bank to receive payment for the rendered service with monies from other MSA members that are not pooled prior to paying the provider and are pooled by the bank, not the MSA, at the time the provider is paid. This exemplary embodiment or another embodiment may further provide queuing, by the provider, an account receivable at a provider bank account for the rendered service to the first member with monies from other MSA members that are not pooled prior to paying the provider and are pooled by the bank, not the MSA, at the time the provider is paid in one of a collective check and a direct funds transfer from the bank. This exemplary embodiment or another embodiment may further provide ranging, by the provider, a time to receive payment for the rendered service to the first member with monies from other MSA members that are not pooled prior to paying the provider and are pooled by the bank, not the MSA, at the time the provider is paid in one of (i) a collective electronic or manual check drawn from the bank and (ii) a direct funds transfer from the bank. This exemplary embodiment or another embodiment may further provide registering, by the provider, that the payment from the bank has been received, wherein the payment is for the rendered service to the first member with monies from other MSA members that are not pooled prior to paying the provider and are pooled by the bank, not the MSA, only at the time the provider is paid in the form of one of (i) a collective electronic or manual check drawn from the bank and (ii) a direct funds transfer from the bank.

In yet another aspect, an exemplary embodiment of the present disclosure may provide a method for sharing for medical treatment expenses between members of an organization comprising: joining an organization so as to be designated a first member of the organization; receiving, by the first member, a notification that a bank account has been created for the first member at a bank; depositing, by the first member, money directly or indirectly into the bank account; receiving, by the first member, a rendered service by a provider; affirming, by the first member, that no portion of the money deposited into the bank account of the first member will be pooled into a common fund or common account controlled by the organization; submitting an expense associated with a rendered service provided to the first member by a provider; and confirming, by the first member, that the expense has been paid with monies from other members of the organization that have not been pool into common fund or a common account controlled by the organization prior to paying the provider. This exemplary embodiment or another embodiment may further provide wherein the rendered service is a medical treatment and the provider is one of a doctor, a nurse, and a medically trained professional, wherein the provider treats the first member as a patient. This exemplary embodiment or another embodiment may further provide submitting, by the first member, the expense associated with the rendered service directly to the organization. This exemplary embodiment or another embodiment may further provide submitting, by the provider, the expense associated with the rendered service to the first member directly to the organization. This exemplary embodiment or another embodiment may further provide effecting a submission of an invoice for the expense to the organization, wherein the submission of the invoice may be from one of (i) the provider, and (ii) the first member. This exemplary embodiment or another embodiment may further provide effecting a generation of an invoice for the expense to the organization, wherein the generation of the invoice may be from one of (i) the provider, and (ii) the first member. This exemplary embodiment or another embodiment may further provide effecting an update for an invoice for the expense to be provided to the organization, wherein the update of the invoice may be from one of (i) the provider, and (ii) the first member. This exemplary embodiment or another embodiment may further provide cooperating, by the first member, with the organization to ensure that the bank receives sufficient information to update a payment file associated with the expense for the rendered service and that the payment file includes information to pull monies from other member bank accounts, without pooling any funds, to pay the expense for the first member. This exemplary embodiment or another embodiment may further provide cooperating, by the first member, with the organization to ensure that the bank receives a method of payment file associated with the expense for the rendered service and that the payment file includes information to pull monies from other member bank accounts, without pooling any funds, to pay the expense for the first member. This exemplary embodiment or another embodiment may further provide cooperating, by the first member, with the organization to ensure that the bank debits payments from other member bank accounts and credits member transaction accounts, and a report of the debits and credits is provided back to the organization that has an option to share the debit and credit information with the first member. This exemplary embodiment or another embodiment may further provide accumulating, by the first member, a plurality of expenses associated with the rendered service for the first member from the provider; and submitting, by the first member, the plurality of expenses to the organization that effects the bank to pay the plurality of expenses with monies from other member bank accounts, wherein the monies from other member bank accounts remain independent, distinct, and are not pooled prior to paying the provider. This exemplary embodiment or another embodiment may further provide aggregating, by the first member, expenses for submission to the organization that the organization will provide to the bank to pay the expenses of the first member with monies from independent and non-pooled bank accounts owned by other members of the organization. This exemplary embodiment or another embodiment may further provide allotting, by first member, funds in the bank account of the first member for expenses submitted to the organization on behalf of other members of the organization; and maintaining funds in the bank account of the first member independent from any collective fund associated with all members of the organization. This exemplary embodiment or another embodiment may further provide appending, by the first member, a digital copy of an expense for the rendered service for the first member, wherein the expense is to be paid by the bank with monies maintained in independent accounts of other members of the organization in response to instructions received from the organization based, at least in part, on the digital copy of the expense for the rendered service. This exemplary embodiment or another embodiment may further provide assigning, by the first member, rights in receivable payments from the bank to the organization, wherein the upon receipt of the receivable payments by the organization, the organization distributes funds to the provider. This exemplary embodiment or another embodiment may further provide authenticating, by the provider, the expense that is to be paid by the bank in response to receiving instructions from the organization; and effecting, by the provider, the bank to pay the provider for the expense of the rendered service with funds from other members maintained in independent accounts. This exemplary embodiment or another embodiment may further provide combining, by the first member, a plurality of expenses submitted to the first member from the provider for payment; and effecting, by the first member, the bank to pay the plurality of expenses with funds from independent and non-pooled funds from other members of the organization. This exemplary embodiment or another embodiment may further provide documenting, by the first member, a plurality of expenses for submission to the organization that are to be paid by the bank in response to the submission. This exemplary embodiment or another embodiment may further provide delivering, by the first member, a digital copy of the expense for the rendered service for payment directly to the provider by the bank with funds maintained in individual and independent member accounts. This exemplary embodiment or another embodiment may further provide documenting, by the first member, a digital copy of the expense for submission to the organization that provides instructions for the digital copy to the bank for the bank to pay the expense with monies from other member bank accounts that are maintained independently without pooling any funds between member bank accounts. This exemplary embodiment or another embodiment may further provide entering, by the first member, the expense into the organization that is to be paid to the provider form the bank. This exemplary embodiment or another embodiment may further provide equipping, by the first member, the organization with digital information representing a bill for the rendered service received by the first member from the provider that is to be paid for by the bank with funds from other members of the organization that are maintained in independent and distinct bank accounts, wherein the funds that pay the bill are not pooled prior to paying the provider. This exemplary embodiment or another embodiment may further provide exchanging, by the first member, information with the organization regarding the rendered service so as to enable the organization to direct the bank to pay a bill for the rendered service with funds from other members of the organization that are maintained in independent and distinct bank accounts, wherein the funds that pay the bill are not pooled prior to paying the provider. This exemplary embodiment or another embodiment may further provide extracting, by the first member, an expense from the prior subsequent to the rendered service, and providing a bill representing the expense directly to the organization so as to enable the organization to direct the bank to pay the bill for the rendered service with funds from other members of the organization that are maintained in independent and distinct bank accounts, wherein the funds that pay the bill are not pooled prior to paying the provider. This exemplary embodiment or another embodiment may further provide facilitating, by the first member, a cooperation between the organization and the provider, either before or after the rendered service, so at to enable the organization to direct the bank to pay a bill for the rendered service with funds from other members of the organization that are maintained in independent and distinct bank accounts, wherein the funds that pay the bill are not pooled prior to paying the provider. This exemplary embodiment or another embodiment may further provide decreasing funds in the bank account of the first member in response to other members of the organization submitting additional expenses to the organization that are to be paid by the bank with funds from the first member that are maintained independently and not pooled with any other funds controlled by the bank. This exemplary embodiment or another embodiment may further provide monitoring, by the first member, a portal hosted by the organization that reflects payments made to the provider for a bill received by the bank with funds from other members of the organization that are maintained in independent and distinct bank accounts, wherein the funds that pay the bill are not pooled prior to paying the provider.

In yet another aspect, an exemplary embodiment of the present disclosure may provide a sharing system providing at least one technical solution to technical problems arising from pooling funds from multiple members of an organization, wherein the sharing system eliminates technical problems by precluding pooling of funds, the system comprising: an organization composed of a plurality of members; a bank responsible for a plurality of independent bank accounts, wherein one bank account is associated with one member of the organization; at least one non-transitory computer readable storage medium having instructions thereon that, when executed by at least one processor, perform operations to segregate funds of the plurality of members such that no funds are pooled into a common account, the instructions further include: open a first bank account in response to a first member joining the organization; effect the first member to fund the first bank account; pay an expense for a rendered service received by the first member with funds from bank accounts of other members of the organization; and pay a second expense for a second rendered service received by a second member with funds from the first bank account. This exemplary embodiment or another embodiment may further provide a specific need of the first member for payment of the rendered service; and payment logic to determine whether the specific need occurs within a pre-determined period of time and the specific need does not depend on moral values of other members. This exemplary embodiment or another embodiment may further provide wherein neither the members, nor the bank, nor the organization generates reserves of funds. This exemplary embodiment or another embodiment may further provide wherein the instructions further include: receive, at the organization, a bill from a provider that provided the rendered service to the first member; direct the bank to pay the provider with funds from other members for the bill associated with the rendered service for the first member, wherein the funds are not pooled. This exemplary embodiment or another embodiment may further provide wherein the funds are not pooled prior to paying the provider. This exemplary embodiment or another embodiment may further provide wherein the instructions further include: accumulate, at the organization, a plurality of bills from a plurality of providers that rendered services to multiple members of the organization over a period of time; instruct, by the organization, the bank to pay at least one bill from the plurality bills with independent and segregated funds from bank accounts of the members not receiving the plurality of bills form the plurality of providers.

In yet another aspect, an exemplary embodiment of the present disclosure may provide a method for a mutual sharing association that complies with government regulations to ensure that the mutual sharing association is not insurance, the method comprising: determining a specific need of a first member of an mutual sharing association (MSA) to be funded with a specific payment of a second member of the MSA; receiving instructions at an automated banking platform from a MSA member application, wherein the instructions pertain to the specific need of the first member to be paid, at least partially, with the specific payment of the second member of the MSA; transferring, via an automated banking platform, funds to a first bank account of the first member from a second bank account of the second member directly and without directive from the second member, without pooling any funds of the second member with funds from other MSA members prior to entering the bank account of the first member; wherein no reserves of funds are built by the MSA or by the a bank hosting the bank accounts of the first member and the second member; and disclosing, via a MSA member application, to members of the MSA that its sharing service is not insurance and does not imply a guarantee of benefits. This exemplary embodiment or another embodiment may further provide wherein members of the MSA do not rate members of the MSA based on moral values, and the method further comprises: transferring funds from the second member to the first member based, at least partially on, a period of time when the first member submitted the specific need to the MSA. This exemplary embodiment or another embodiment may further provide opening the first bank account at the bank in response to the first member joining the MSA; and opening the second bank account at the bank in response to the second member joining the MSA. This exemplary embodiment or another embodiment may further provide effecting the second member to fund the first bank account. This exemplary embodiment or another embodiment may further provide paying the specific need of the first member with funds from bank accounts of other members of the MSA. This exemplary embodiment or another embodiment may further provide receiving, at the MSA, a bill from a provider that provided a rendered service to the first member that generated the specific need. This exemplary embodiment or another embodiment may further provide directing the bank to pay the provider with funds from the first bank account of the first member that received at least one direct deposit from the second bank account of the second member. This exemplary embodiment or another embodiment may further provide accumulating, at the MSA, a plurality of bills from a plurality of providers that rendered services to multiple members of the organization over the period of time. This exemplary embodiment or another embodiment may further provide instructing the bank, by the MSA, to pay at least one bill from the plurality bills with independent and segregated funds from bank accounts of the members not receiving the plurality of bills form the plurality of providers.

In yet another aspect, an embodiment of the present disclosure may provide a non-insurance system for sharing costs between members of an organization comprising: a first member of the organization having a first bank account maintained at a bank; a second member of the organization having a second bank account maintained at the bank, and wherein the second bank account is independent from the first bank account; a specific need of the first member that is to be paid with funds of the second member; sharing logic maintained by the organization to direct the bank to transfer funds directly from the second bank account to the first account subsequent to the organization learning of a rendered service performed by a provider and received by the first member; a digital pathway for funds from the second bank account to transfer to the first bank account without directive of the second member, wherein the digital pathway is independent and does not merge with other digital pathways along which funds from other members of the organization move between bank accounts. This exemplary embodiment or another embodiment may further provide notification logic to notify members of the organization that the non-insurance system is not insurance and does not imply a guarantee of benefits to pay for the rendered service. This exemplary embodiment or another embodiment may further provide at least one technical solution to technical problems arising from pooling funds from multiple members of an organization, wherein the non-insurance system eliminates technical problems by precluding pooling of funds prior to the first member receiving the rendered service from the provider. This exemplary embodiment or another embodiment may further provide an expense that arises as a result from the rendered service that was rendered to the first member by the provider; a receipt of funds by the first member from the second member that is effectuated in response the expense; and wherein funds from the second member move along the digital pathway. This exemplary embodiment or another embodiment may further provide sharing instructions coupled with the sharing logic, the sharing instructions including digital data indicating that funds from the second bank account of the second member need to be shared with the first member; and funds of the second member that move along the digital to transfer funds from the second member to the first member, wherein the digital pathway is independent and distinct from other pathways associated with other members of the organization. This exemplary embodiment or another embodiment may further provide wherein member funds do not enter a common pool such that no funds are shared with other members prior to the specific need arising. This exemplary embodiment or another embodiment may further provide direct member-to-member transfers between a plurality of individual bank accounts that remain segregated and independent from other bank accounts of members of the organization.

In yet another aspect, an embodiment of the present disclosure may provide a system and method is provided that effectuates member-to-member sharing of funds between bank accounts maintained at a bank. The sharing occurs subsequent to one of the members of the organization receiving a rendered service from a provider. This results in a specific sharing need that the organization is aware of. The organization directs the bank to transfer funds from one member to the member in need of paying the expense associated with the rendered service. The money from the other members that is deposited into the receiving member's account is then distributed to the provider as a payment for the rendered service. The money may be distributed directly from the bank, or the money may be provided to the organization that directs the payment to the provider.

### BRIEF DESCRIPTION OF THE SEVERAL VIEWS OF THE DRAWINGS

A sample embodiment of the disclosure is set forth in the following description, is shown in the drawings and is particularly and distinctly pointed out and set forth in the appended claims. The accompanying drawings, which are fully incorporated herein and constitute a part of the specification, illustrate various examples, methods, and other example embodiments of various aspects of the disclosure. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 is a diagrammatic view of a system for implementing cost sharing between members in accordance with the present disclosure.

FIG. 2 is an operational schematic view of the system of FIG. 1 depicting cost sharing directly between members.

FIG. 3 is an operational schematic view of a sharing model in accordance with the present disclosure.

FIG. 4A is a flowchart depicting an exemplary method or process of operating a system in accordance with the present disclosure.

FIG. 4B is a flow chart depicting an exemplary method or process of member-to-member transfers.

FIG. 5 is a flowchart depicting an onboarding process in accordance with the present disclosure.

FIG. 6 is a flowchart depicting a funding process in accordance with the present disclosure.

FIG. 7 is a flowchart depicting a sharing process in accordance with the present disclosure.

FIG. 8 is a flowchart depicting a conversion onboarding process in accordance with the present disclosure.

FIG. 9 is a flowchart depicting a conversion funding process in accordance with the present disclosure.

Similar numbers refer to similar parts throughout the drawings.

### DETAILED DESCRIPTION

As depicted throughout the figures, a system and method for cost sharing is provided herein. The system for cost sharing is shown generally at 10. System 10 includes a data center 12, a firewall 14, an application server cluster 16, a database cluster 18, a secondary data center 20, a backup application server 22, and a replicated slave cluster 24.

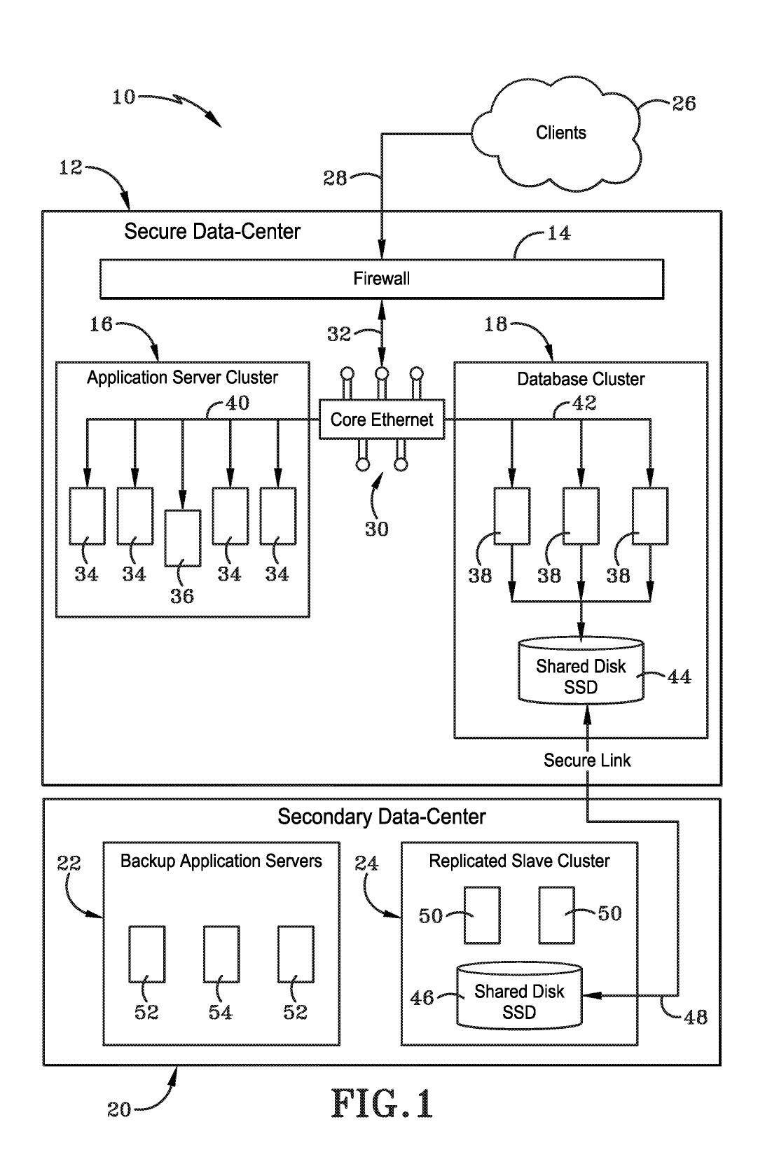

FIG. 1 depicts clients generally at 26. Clients 26 are in communication with the data center 12 via line 28 representing an electrical communication or an electrical network, such as the Internet or other communication networks. Line 28 representing the electrical connection between clients 26 and the data center 12 is in operative communication with firewall 14. Firewall 14 is electrically connected with a core Ethernet 30 via line 32. The core Ethernet 30 is in electrical communication with the application server cluster 16 and the database cluster 18. Within the application server 16 are a plurality of servers shown generally at 34. In addition to the servers 34, a swarm manager 36 is electrically connected with the servers 34. The database cluster 18 may include a plurality of databases 38 in electrical communication with the core Ethernet 30. The servers 34 and the swarm manager 36 may be in electrical communication with the core Ethernet via line 40. The databases 38 may in electrical communication with the core Ethernet via line 42. At least one of the databases 38 is in electrical communication with a storage device 44. Storage device 44, in one particular embodiment, may be a non-transitory computer readable storage medium. Storage device 44 may be in electrical communication with a secondary storage device 46 via a secure link line 48. Secure link line 48 operatively couples the data center 12 with the secondary data center 20. More particularly, the secondary storage 46 is part of the replicated slave cluster 24 within the secondary data center 20. Replicated slave cluster 24 may include at least one secondary database 50. Backup application server 22 may include at least one secondary server 52 and at least one secondary swarm manger 54. The backup application servers 52 and backup or secondary swarm manager 54 are part of the backup application servers 22 within the secondary data center 20. In one particular embodiment, the secondary application server 22 may be in operative communication with the replicated slave cluster 24.

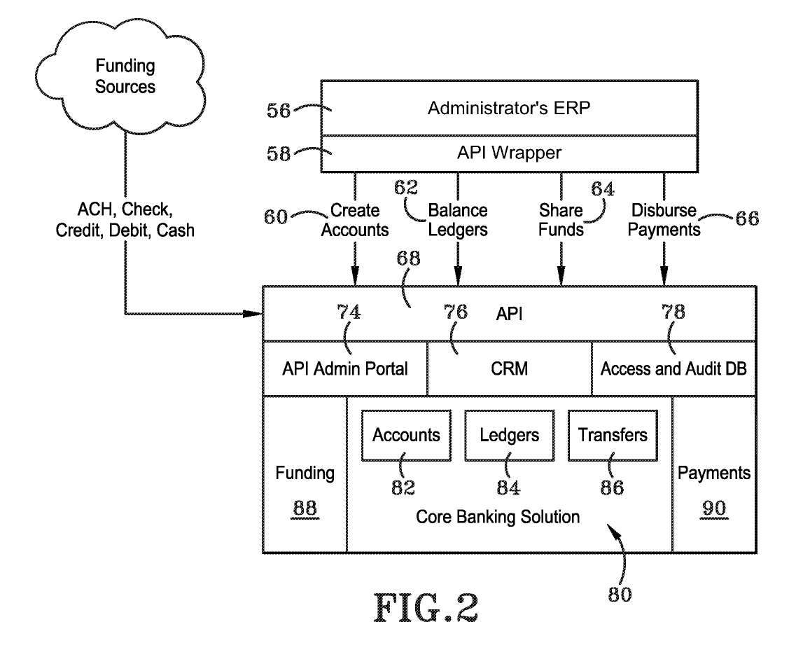

FIG. 2 depicts a diagrammatic operation of the system 10. More particularly, the functionality of system 10 is represented by the diagrammatic flow chart depicted in FIG. 2. An administrator's ERP 56 is in operative communication with an application program interface (API) wrapper 58. The API wrapper 58 enables the creation of accounts 60, balance ledgers 62, sharing of funds 64, and disbursement of payments 66. The creation of accounts 60, balance ledgers 62, sharing of funds 64, and disbursements of payments 66 are directed and in communication with a second API 68. The second API 68 receives input via line 70 of ACH, check, credit, debit, or cash from funding sources 72. The second API 68 is in operative communication with an API administration portal 74, a CRM 76, and an access and audit database 78. A core banking solution 80 includes accounts 82, ledgers 84, and transfers 86. The core banking solution 80 is in operative communication with the API administration portal 74, the CRM 76, and the access and audit database 78. Collectively, the core banking solution 80 operates to establish funding 88 and payments 90.

With continued reference to FIG. 2, in order to allow for flexibility in the back end support for multiple banking platforms and/or allow for upgrades in the banking platform as growth dictates, the present disclosure provides a system that is built around a coupled API pattern separating the customers 26 from underlying specifics of the banking platform. The core banking solution 80 may be provided by a third party software, such as, by way on non-limiting example, Jack Henry Software. The API 68 serves as a single point of entry into the core banking solution 80 as well as it allows for provisioning and utilization of the core banking solution 80 to happen in a central manner. The API 68, in one particular embodiment, is secured with an industry standard technology that allows easy integration with software tooling and security systems of any customer. In one particular embodiment, this enables the API 68 to utilize open ID connect tokens and enables the possibility of an implementation of a federated identity and access management product, such as PING Federate. The API wrapper 58 is a programmatic solution for accessing and using the API 68. The API wrapper 58 allows for a greater deal of control as to how the API 68 is used, as well as control for who uses it. Additionally, by providing some implementation to the customer, the API wrapper 58 eases the implementation burden for the technology solution of the present disclosure. In one particular embodiment, JAVA Script or JAVA or Dot Net are utilized. The administration portal 74 can be utilized and developed as a portal to administer the API access and review audit information. The CRM 76 can leverage the core banking platform with a solution, such as sales force, and there needs to be a degree of management of the customers of the API, the healthcare sharing organizations or MSAs, on top of the management of the individual members within each organization. This allows a mechanism for tracking accounts payable, accounts receivable, gains and losses, and other operational functions of the business of administering this system. The access and audit database 78 assists in billing as well as any regulatory inquiries. The access and audit database 78 is different and independent than the recording of the financial transactions implemented by banking platform of the system 10.

The core banking solution 80 may be a software service or software solution currently available by vendors, such as Jack Henry. The core banking solution 80 provided by a software service may include a function that can specify which member account funds will be withdrawn from and specify an explanation of what that check is for in detail along with the check or ACH transaction. In accordance with the present disclosure, funds are not moved to a distribution account or a collected or pooled account before money is transferred to the providers. The creation of accounts through an online and paper enrolment process is another portion of the core banking solution 80. Customers should be able to complete any and all steps to create a bank account, sign documents, and assign any powers of attorney, if necessary, to the healthcare sharing organization implemented by system 10 to enable the sharing of funds and distribution of payment without any pooling or collection of member funds in a central account. Each account 82 can have an identification code, an account number, a routing number, a name, and a creation date. Each ledger 84 includes an identification code, a list of transactions, an account owner or account owner identification, and a current balance. In one particular embodiment, there may be multiple interfaces depending upon whether there is a payee or a transaction string.

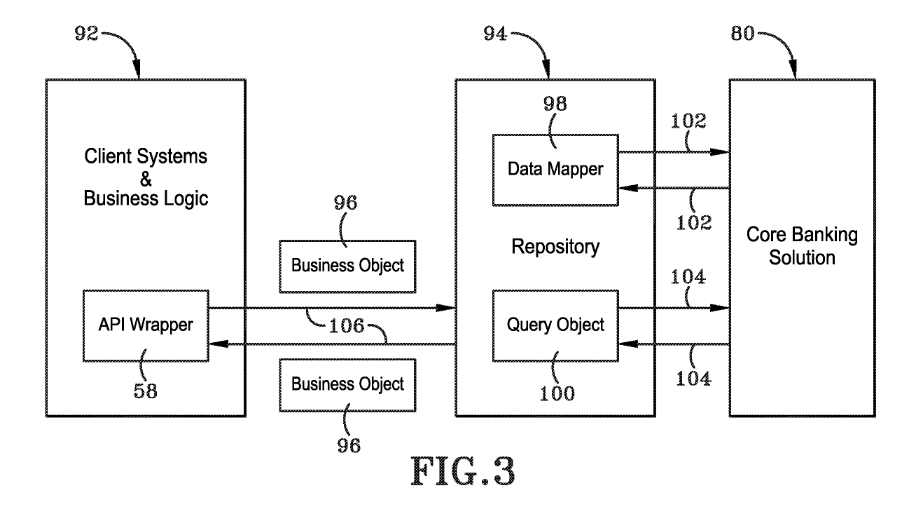

FIG. 3 depicts a representative exemplary schematic model. The core banking solution 80 is built from the design of a core banking platform or third party solution, such as Jack Henry software. The client systems and business logic 92 are built and driven by the customers. The repository 94, the API wrapper 58, and the business objects 96 are constructed against models developed as part of the system 10 in accordance with the present disclosure to improve cost sharing between parties. Core banking solution 80 is in operative communication with a data mapper 98 and a query object 100 which are part of the repository 94. The API wrapper 58 is shown within the client systems and business logic 92 because it is driven by the customers or clients 26.

As depicted in FIG. 3, the core banking solution 80 is in operative communication to transfer data with the data mapper 98 in the repository 94 via two-way transmission lines 102. Additionally, core banking solution 80 is in operative communication with the query object 100 in repository 94 via two-way communication via transmission lines 104. The repository 94 is in operative communication with the API wrapper 58 which may be portion of the client system and business logic 92 as indicated via two-way transmission lines 106.

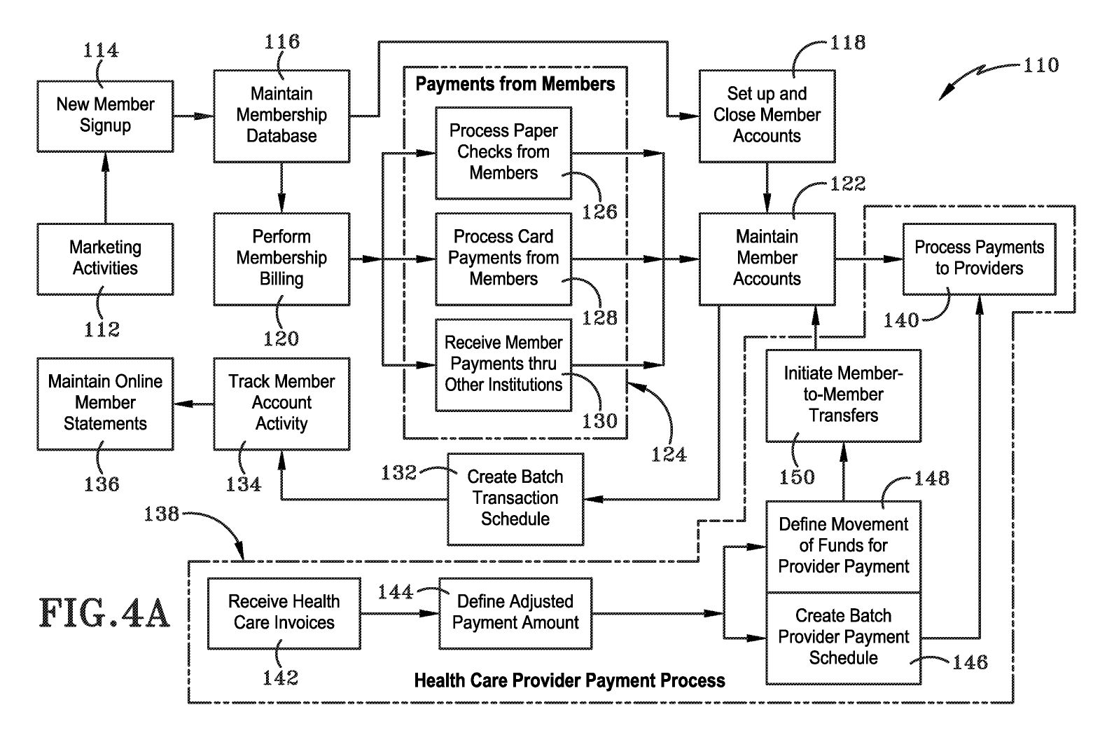

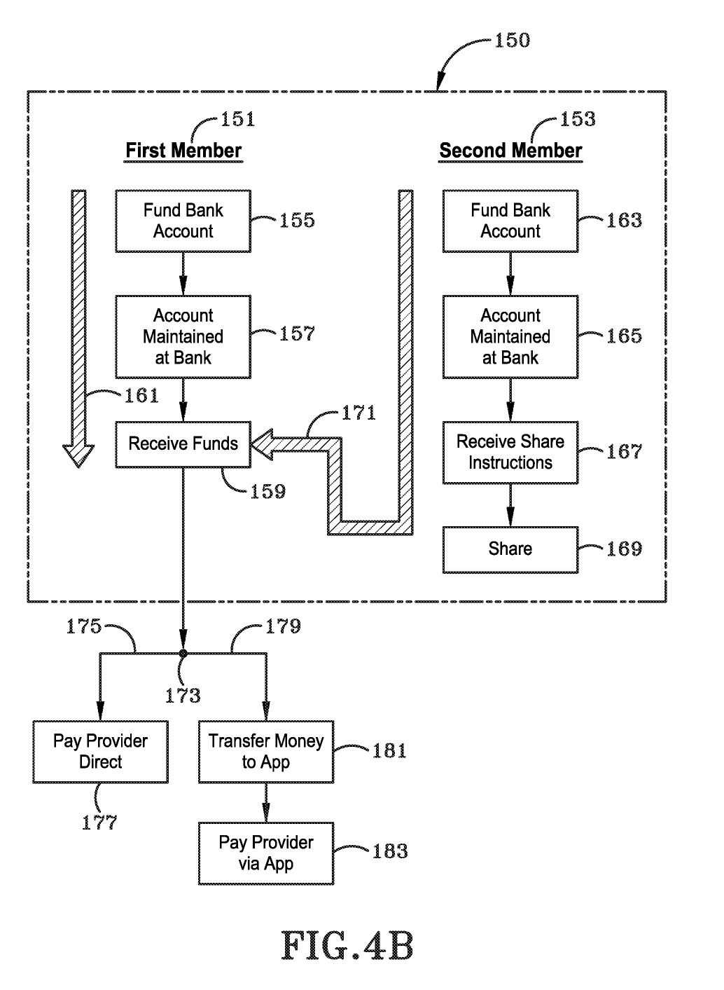

FIG. 4 depicts an exemplary flowchart of the method of operation for system 10 to implement cost sharing between members of a group or organization. The schematic of the method is shown generally at 110. The method 110 begins with marketing activities 112 performed by a company. The marketing activities should include various activities as one having ordinary skill in the art would understand to generate leads and pursue potential members to join the cost sharing initiative. After the marketing activities have been performed at 112, a new member is signed up by the company, which is shown generally at 114. After a new member has been signed up at 114, the membership database is maintained and updated at 116 to reflect the new member signup. From the database at 116, a new bank account may be set up at 118. Alternatively, the database at 116 can perform membership billing, which is shown at 120. With continued reference to setting up of bank accounts at 118, this may be performed at a bank that is wholly owned by the company or at a partner bank that is in a cooperative relationship with the company. The bank may maintain the member bank accounts at 122. The performance of membership billing at 120 may be considered a “share box” which is able to collect automatic ACH data pertaining to the bank accounts which members will share directly with other members without causing any monies to be pooled or collected in a general account. The payments from members is shown generally at 124. Within the payments for members 124 are various steps that are in communication with the performance of membership billing at 120. Particularly, processing paper checks from members occurs at 126. Processing credit card payments from members occurs at 128 and receiving membership payments from other institutions, such as other bank direct deposits, occurs at 130. After the payments made from the members at 124 is complete, the payments are then updated in the member accounts which are maintained at 122. From the maintenance of the member accounts at 122, a batch transaction schedule is created at 132. After the batch transaction schedule is created at 132, the member account activity is tracked at 134 and online member statements are maintained at 136.

The maintenance of member accounts at 122 is coupled with the provider payment process 138. In one example, the provider is a healthcare provider, such as a doctor or hospital. The processing of the payments to providers is shown generally at 140 and is connected with the maintenance of member accounts at 122. The healthcare provider payments process 138 receives healthcare invoices at 142 and defines an adjusted payment amount at 144. Then, a batch provider payment schedule may be created at 146 or the movement of funds for provider payment may be defined at 148 which is connected to an initiation of member to member transfers at 150 which is connected with the maintenance of member accounts at 122. After the movement of funds created by 148, the processing may occur to provide payments to medical providers at 140. Regardless of how the processing of payments to medical providers is accomplished at 140, it is established at step 122 that the member-to-member accounts transfer and effectuate the sharing of costs that are input into the system at 142. In every scenario, no money is pooled in a general account for later disbursement. Rather, member-to-member transfers at 150 occur to effectuate the payments to providers at 140. State otherwise, funds of the first member travel along a first pathway and funds of the second member travel along a second pathway. The pathways remain independent such that only money from one member flows along a respective pathway and there are no unions along the pathways. However, some pathways may terminate at another pathway. For example, a first pathway may extend from a funding source of the first member (such as a credit card or an external bank account) to the first bank account maintained by the bank. A second pathway may extend from a funding source of the second member to the second bank account maintained by the bank. The second pathway may further extend from the second bank account to the first bank account and terminate at the same. In this instance, money from the second member can travel independently along the second pathway directly from the second bank account to the first bank account without pooling with any funds along the pathway. It is to be understood that the number of pathways associated with movement of funds should be equal to the number of members of the MSA such that funds from each member remain independent from those of other members.

FIG. 4B depicts an exemplary model of the member-to-member transfers 150. In this example, a first member 151 of the MSA and a second member 153 of the MSA may maintain respective independent bank accounts at the bank. Funds will travel along independent pathways associated with each of the members 151, 153, respectively. The first member 151 may fund a first bank account at 155. The first bank account is maintained at the bank, which is shown generally at 157. When a need for an expense arises that results from a service that was rendered to the first member 151 by a service provider, the first member 151 may receive funds at 159 from other members of the MSA. In this particular example, the first member 151 receives funds at 159 from the second member 153. A first pathway 161 is defined as the movement of funds or monies for the first member 151 between the steps of funding the bank account 155, maintaining the bank account 157, and receiving funds 159.

The second member 153 funds a second bank account at 163. The second bank account is maintained by the bank, which is shown generally at 165. The MSA application receives sharing instructions associated with the services rendered to the first member 151, which is shown generally at 167. The sharing instructions contain digital data indicating that funds from the second bank account of the second member 153 need to be shared, which is shown generally at 169, with the first member 151. The funds of the second member 153 move along a second pathway 171 to transfer funds from the second member 153 to the first member 151, which is defined between the steps of funding the bank account 163, maintaining the second bank account 165, receiving the share instructions 167, and the sharing of the funds 169. The second pathway 171 is independent and distinct from the first pathway 161. Stated otherwise, the first and second pathways associated with the flow of member funds do not enter a common pool such that no funds are shared with other members prior to receiving the share instructions at 167. This is in contradistinction to an insurance model which would receive funds from the members first and then pool them directly into a common account, and then receive the invoices for the services rendered, such as seeing a doctor, and then pay the medical provider directly. In this particular example, the second pathway 171 terminates at the bank account of the first member 151 at the step of receiving funds 159.

After the first member 151 receives the shared funds from the second member 153, the options for the first member 151 split at node 173. A first option, which is identified by leg 175, enables the first member 151 to pay the provider of the rendered service directly, which shown generally at 177. The second option, which is a more preferred option, enables funds to travel along leg 179 in which the funds that were received from the second member 153 to pay for the first member's 151 service that was rendered is transferred to the MSA application, which is shown generally at 181. Then the MSA application pays the provider directly, which is shown generally at 183. When the MSA application pays the provider, which is shown at 183, the payment may be effectuated in a number of ways which are described in greater detail below. In some instances, the MSA application that pays the provider at 183 can either mimic the look and feel of a check associated with that of the first member 151 so that the provider believes that the member is paying them directly. Alternatively, the check which is paid to the provider at 183 may include the logo and identification of the MSA application. Further aspects of the member-to-member transfers or shares are described in greater detail below with respect to the subsequent figures.

With continued reference to FIG. 4B, and other figures, it is shown that a method of sharing costs between multiple parties, such as first member 151 and second member 152, may include one or more of the following processes. For example, a method may provide maintaining, by the bank, a plurality of individual bank accounts that remain segregated and independent from other bank accounts of members of the MSA. Then, receiving funds, deposited by one of a mutual sharing association (MSA) application and the first member 151 of the MSA, into a first bank account of the first member that is maintained by the bank, which was shown generally at 155, wherein the first member and the MSA application are different entities, and wherein the funds travel along the first pathway 161 which can also extend from the MSA application to the first bank account. This method may further provide receiving a notification that a provider has rendered a service to the first member 151. Then, receiving data, in a processor, of a payment to be paid in exchange for the service that was previously provided by the provider and received by the first member (i.e., sharing instructions 169). Then, transferring funds directly from a second bank account of the second member 153 that is maintained by the bank along the second pathway 171 in response to a provider payment schedule or other digital data provided to the bank by the application, wherein the second pathway 171 is independent from the first pathway 161. Then, paying the provider with funds from the second member.

Some other exemplary methods include wherein paying the provider is accomplished by funds from the second member 153 held in the second bank account moving solely along the second pathway 171 and not along the first pathway. In this scenario, the second pathway 171 would not terminate at step 159. Rather, the second pathway 171 could extend independently down to paying the provider directly at 177 or through the MSA application which is shown at 183. Further, paying the provider may be accomplished by funds from the second member transferred along the second pathway into the first bank account, and the funds from the second member are automatically withdrawn by the bank from the first bank account.

Additional aspects of the method(s) of FIG. 4B include receiving, in the processor, monthly invoices generated by the MSA application containing data associated with services performed by the provider to be paid by the bank for other members of the MSA. Receiving the monthly invoices may be accomplished by a transferring from a file generated by the MSA application; wherein each file generated by the MSA application is associated with movement of funds between bank accounts along respective independent pathways such that funds are not pooled. Then, receiving data associated with a method of payment for the bank to pay the provider with funds of the second member from the first bank account of the first member, wherein the second pathway extends from the second bank account to the first bank account; and moving funds along the second pathway 171 into the first bank account, wherein only funds from the second member 153 move along the second pathway such that funds from the second member remain independent and segregated from other funds traveling along other pathways of other members that terminate at the first bank account that receives funds at 159. The bank may further be receiving updated invoices from an accounts receivable file in the MSA application, wherein the invoices travel along a common pathway from the MSA application to the bank. Additionally, the bank may be receiving a payment method file from an accounts receivable at the MSA application, wherein the payment method file travels along the common pathway.

Further aspects of the method(s) of FIG. 4B include paying the provider, directly from the bank, with funds credited to the first member from the transaction of the second member. And, paying the provider, directly from the bank, with the monies of the second member 153 in response to the received data of the payment to be paid in exchange for the previously provided service from the provider received by the first member 151, wherein the funds from the second member 153 that move along the second pathway 171 remain independent from other pathways along which funds from other members move. This method or another exemplary method may also provide for receiving, at the bank, invoices generated by the MSA application in response to the first member submitting a bill to the MSA application; and identifying the bill to be paid for services provided by the provider to the first member, and initiating a payment for the bill to be sent directly from one of the second bank account and the first bank account containing funds sourced from the second member 153. In some aspects, the method may provide for paying the provider, directly from the bank, with monies from the second member 153 moving along the second pathway 171 for the services provided to the first member 151 in response to receiving, at the bank, data associated with invoices supplied by the provider.

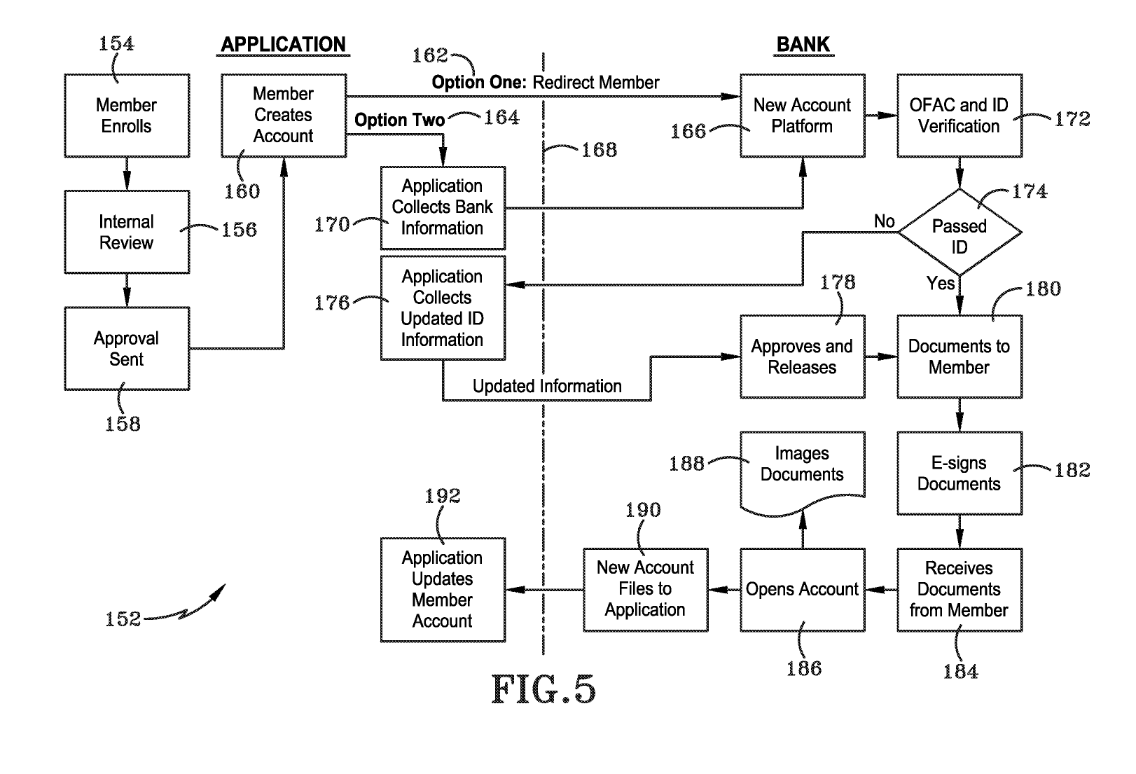

FIG. 5 depicts an onboarding process shown generally at 152. The onboarding process or method 152 begins when a member enrolls at 154. After the member enrolls at 154, an internal review is performed at 156 to determine whether the member is qualified to participate in the sharing association or MSA. In some instances, an exemplary qualification relates to religion. Thus, one exemplary MSA may be formed from person of the same religion, such as Christianity, Catholicism, Islam, or Judaism, or any other religion. After the internal review at 156, an approval is sent at 158, if the new member qualifies, and the new member creates an account at 160. From the account creation at 160, there are two options in order for a new account to be created. A first option is indicated at 162 and a second option is indicated at 164. With the first option 162, a new account can be created at 166 which is provided a unique bank account number. The creation of the new account at 166 is on an opposite side of an imaginary line 168. The imaginary line 168 represents different side of the transaction wherein the boxes to the left side of imaginary line 168 represent objects and actions that can be handled by the application software and the platforms, and boxes and actions located to the right of the imaginary line 168 by a banking platform. If following the second option 164, the application may collect the bank information at 170 and forward the same to the bank crossing over the imaginary line 168 and providing the same to the bank in order to create a new account at 166. After the new account has been created at 168, a verification process is implemented at 172. The verification process by the bank at step 122 then determines if the proper identification information is sufficient at 174. If the information at 174 is insufficient, then the updated information is collected by the application software at 176 which crosses the imaginary line at 168. After the updated information has been collected, it may be resent to the bank for approval and releases at 178. After the information has been removed, approved and released, the documents may be provided to the member at 180. Note, if the identification information was sufficient at step 174, the documents may be sent directly to the member at 180. After the documents have been provided to the member, the member may sign them electronically at step 182. The bank receives the e-signed documents from the member at 184. Once the documents from the member have been received, the bank may open the account at 186 which in some embodiments the documents may be imaged at 188. Once the account has been opened at 186, the new account files are forwarded to the application at 190. The files that have been forwarded to the application at 190 are updated and reflected in the member account in the application at 192.

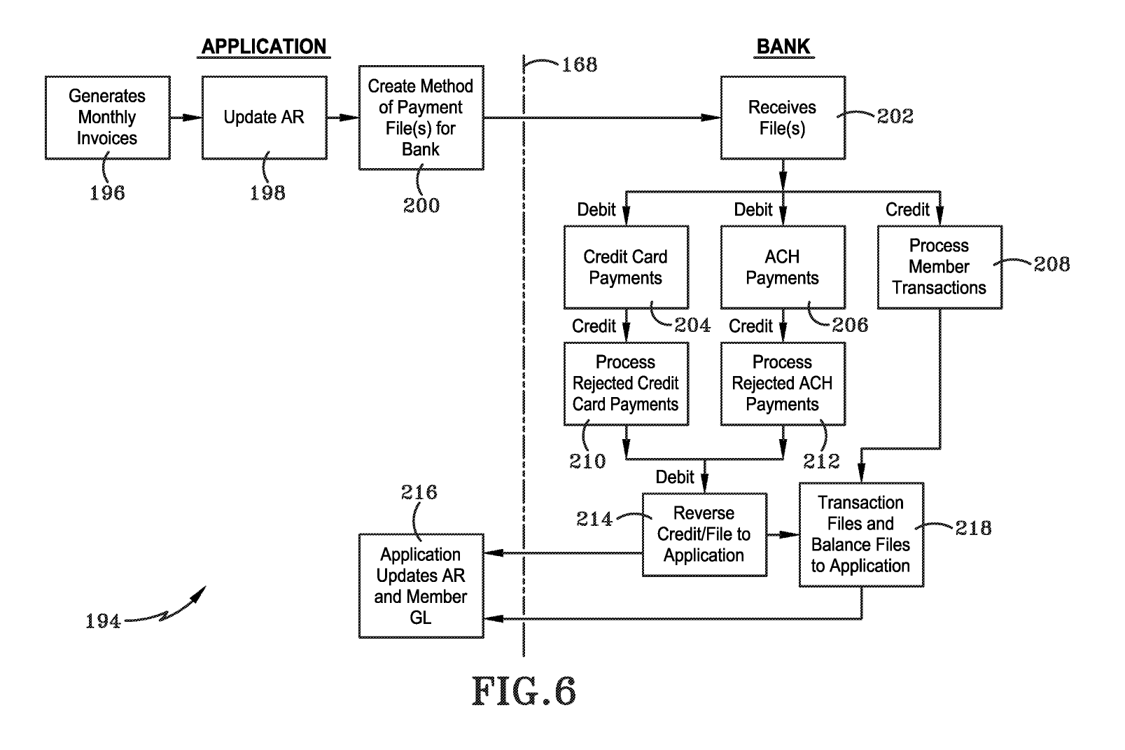

FIG. 6 depicts an exemplary aspect of a funding process generally at 194. The funding process 194 includes a similar imaginary line 168 representing the divisions occurring between the application and a bank. Within the application, monthly invoices are generated at 196. The invoices may depend on costs for services that were rendered to a member. The invoices that were generated are then updated accordingly in the accounts receivable at 198. Then, a method of payment files for later transfer to the bank is created at 200 from the updated accounts receivable at 198. The method of payment files are then sent to the bank that receives the files at 202. After the bank receives the payment files at 202, the information may debit credit card payments at 204, or it may debit ACH payments at 206, and credit member transactions at 208. However, in another particular embodiment, the bank debit the various payment options prior to receiving the payment files 202. When the credit card payments are debited at 204, a credit may be processed for rejected credit card payments at 210. Similarly, after an ACH payment has been debited at 206, a process may be credited for rejected ACH payments at 212 which then collectively reverse the credit file to the application through a debit function at 214. From the reverse credit file at 214, the information may be sent to the application which may update accounts receivable and the member general ledgers at 216. The reverse credit/file at 214 may further provide information to the transaction files and the balance files stored in the bank at 218. The transaction files at 218 may be further sent to the application to update the accounts receivable and the member general ledger at 216.

With continued reference to FIG. 6, and more particularly the monthly invoices that are generated at 196, the manner in which the monthly invoices may be generated can occur by receiving information from a secondary source so as to identify a bill or cost that needs to be paid through the shared effort of the members who are a group or a portion of the application. In one particular embodiment, it is possible to generate the monthly invoices at 196 responsive to the members directly submitting their bills that they desire to be shared amongst the other members directly to the application. Alternatively, in other scenarios, a provider of a service that has the foreknowledge that the member is part of the group or otherwise subscribes to the application may send a bill directly to the application. Alternatively, there may be other possible ways in which the application learns or becomes aware of the monthly invoices that the member desires to share and have paid by the other members directly. For example, the application may include a predictive algorithm or a predictive method configured to learn patterns relating to past invoices and predict an invoice amount for a future time period. Additionally, the application may include a self-referential table including the data from the plurality of members to determine how the direct member-to-member sharing occurs.

In some instances, the application can receive the invoices that are to be paid and data associated with the same directly from the provider through electronic means or other electronic communications. However, it is entirely possible that the system may be an analog version in which the users of the application may directly provide hard copies or other paper copies of the bills or invoices, which are to be cooperatively shared and paid by the application, through a mail carrier.

Subsequent to receiving the monthly invoices, which are generated at 196, the invoices may be updated in an accounts receivable which is shown generally at 198. The manner in which the invoices may be updated in the accounts receivable can occur through a system or process or algorithm that automatically updates the accounts receivable responsive to receiving the invoices. However, it is entirely possible for the accounts receivable at 198 to be updated in a manual manner such that the invoices are reviewed singularly to confirm the correctness and authentic nature of the bill of the provider or, in a predictive manner. It is envisioned that it would be much faster and simpler to perform bulk batch review in order to update the accounts receivable in a single computer transaction. The updates may occur at a time that is convenient for the operator of the application so as to reduce downtime on the system. However, it is also entirely possible for the system to run updates in the background thereof such that no downtime is needed when the process is updated. This allows users of the application to experience no significant lag or delay in the system, as perceived by the user, during the update process. Thus, there may be an instance when the accounts receivable is being updated where the application shows a first amount at a first time and a second amount at a second time shortly thereafter. It should be further noted that it will be common in the application where the users thereof have no access to the accounts receivable and are merely provided a detailed summary page that would maintain the accounts receivable information in a backend of the application not accessible to a common user without an authorization access. The authorization access may be exclusively limited to an IT person or other administrators or owners of the application and purposely withheld from members of the MSA association without some prior authorization.

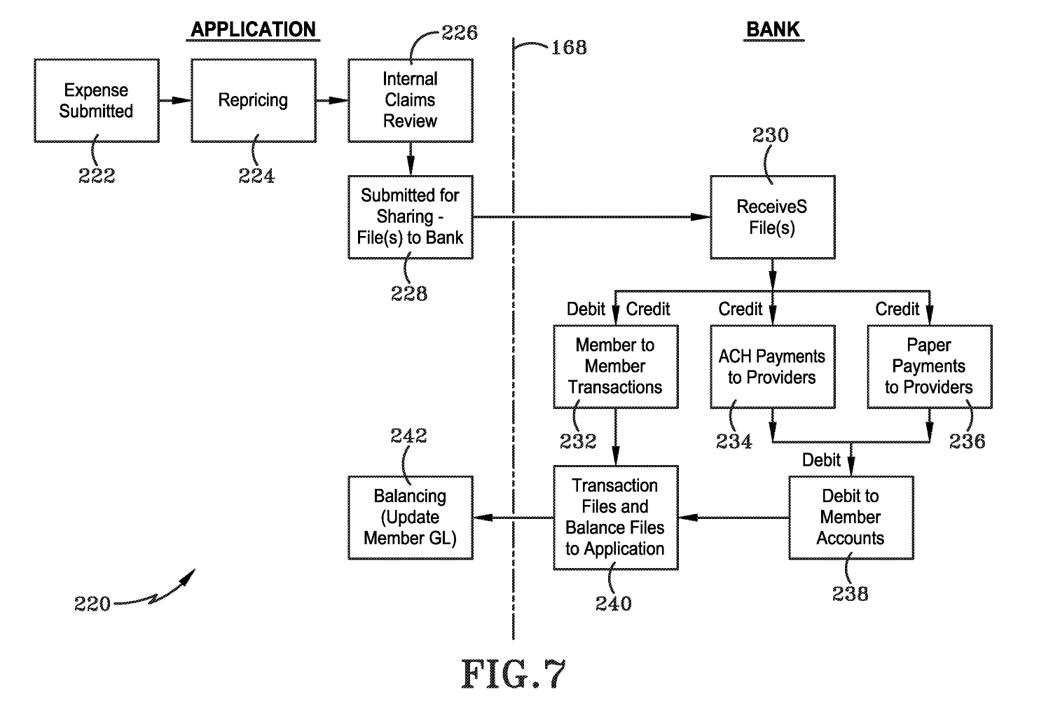

FIG. 7 depicts a sharing model or sharing process in accordance with the present disclosure shown generally at 220. The sharing process 220 effectuates direct member-to-member sharing between an application hosting member accounts and a bank hosting corresponding member accounts segregated by an imaginary line 168. To initiate the sharing process 220, an expense is submitted by one of the members at 222. After the expense has been submitted at 222, a repricing model may occur at 224 in order to reduce some of the costs of the initially submitted expense. Some of the repricing at 224 may include an adjustment of a medical provider's bill. More particularly, the repricing at 224 may include reducing the amount of payment for which the member is liable. After the repricing has been accomplished at 224, an initial claims review may be performed by the application. The internal claims review at 226 then submits the information for sharing by providing the files to the bank at 228. The files that are shared with the bank at 228 are received by the bank at 230. Once the bank receives the files at 230, the information may respectively debit and credit direct member-to-member transactions at 232. More particularly, the member account for whom the expense was submitted at 220 may be credited by equally debiting one or more of another different member's account directly. More particularly, the manner in which the member-to-member transaction occurs is direct such that no portion of the debiting member money is ever sent to a pool or common fund or common account. Rather, it is a direct share occurring in the bank between member-owned accounts. After receiving the files at 230, the bank may further provide the medical providers or other providers with ACH payments in the form of a credit at 234. Additionally, paper payments may be sent to the providers as indicated at 236 which is also denoted as a credit to the providers. After the ACH payments 234 and the paper payments 236 are sent to the providers, a debit to member accounts may occur at 238. From the member-to-member transaction at 232 or from the debit to the member accounts at 238, the transaction files and the balance files are recorded in the bank at 240 and are sent to the application for balancing in order to update the member general ledger at 242. In accordance with one particular embodiment, the member-to-member transaction at 232 may include the transferring, directly and without pooling funds into a shared account, of funds from a second bank account of a second member that is maintained by the bank in response to a provider payment schedule provided to the bank by the application, and paying the provider with monies from the second member. In some instances, paying the provider is accomplished by monies from the second member held in the second bank account. In other instances, paying the provider is accomplished by monies from the second member held in the first bank account.

With continued reference to FIG. 7, and more particularly the initiation of the sharing process 220 by submitting an expense at 222, the manner in which the member may submit the expense can be accomplished in a plurality of different ways. For example, the member may submit an expense to the application via electronic or analog means. In this instance, a member may e-mail an expense or mail the expense to the application for submission as repayment from other members directly. It is to be entirely understood that a representative or other authorized person may submit the expense on behalf of the member. For example, if a husband and wife have shared medical expenses, and the husband is a member, it is possible for the wife to submit the expense on behalf of the husband. Furthermore, the timing of when the expense is submitted may vary. For example, it is common that the expense will be submitted after the service has been provided by the provider. In such an instance, the member will have already received the benefit of the service and may submit the expense to the other members for repayment. However, it is entirely possible for the system or sharing model or sharing process 220 to enable the member to submit an expense 222 that has not yet been received. For example, if a member is requesting a preauthorization expense for a service to be rendered in the future, the expense may similarly be submitted at 222. In this instance, the application may allow for a payment between members to share a future cost. In one scenario that is envisioned may be when the member receives a quote for a service that requires an upfront payment by the service provider. The user may submit the proposed expense to the other members indicating that the service is to be provided in the future, however, payment is required upfront in full.

With continued reference to FIG. 7, the repricing 224 may occur subsequent to the application receiving the expenses which were submitted at 222. In one particular embodiment, the repricing may occur within a model consistent with which members of the application have agreed to pay for certain services. In another particular embodiment, the repricing may occur in a manner unbeknownst to the members and accomplish a price reduction through a negotiation between the application or process or system and the service provider. In one particular example, application may negotiate with the service provider, especially when the service provider is a doctor or hospital or other medical facility providing medical services, in order to reduce the rates for services that the provider is willing to accept in exchange for payment thereof. For example, if a dentist normally changes $300.00 for a traditional dental cleaning, the application may negotiate with the service provider (i.e., dentist) to reprice 224 the dental services to a lower rate, such as $150.00. In one particular embodiment, the repricing occurs subsequent to the expenses being submitted at 222. However, it is entirely possible for the pre-pricing to occur prior to expenses being submitted. In this scenario where the repricing 224 occurs at a different interval in time, the negotiated values for the services provided would be agreed to between the application and the service provider beforehand. This may occur in a scenario where the service provider becomes a recommended provider or an in-network provider as one having ordinary skill in the art would understand.

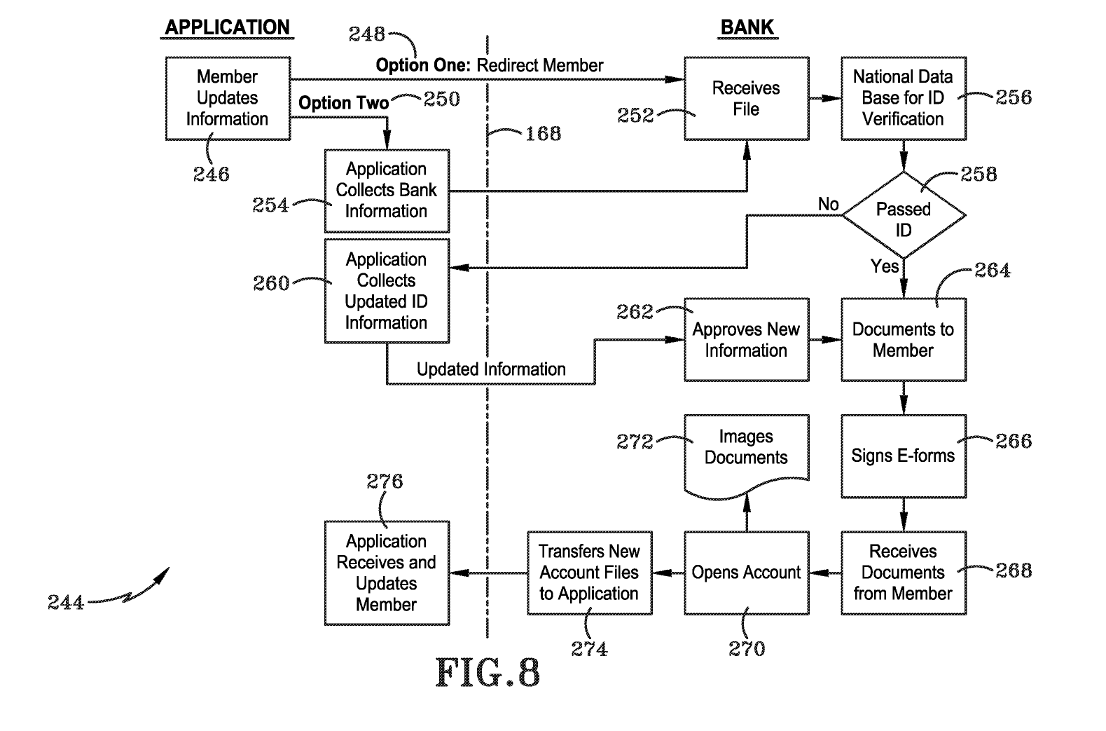

FIG. 8 depicts a conversion onboarding model generally at 244. The conversion onboarding process 244 begins with the member updating information at 246. With onboarding, after a member has updated information at 246, they may proceed down a first option 248 or second option 250. With the first option 248, the application may redirect the member to the bank which receives the member file at 252. In the second option 250, the application can collect the information that the bank will need at 254 and then transfer that same information to the bank which receives the files at 252. After the bank has received the files at 252, the bank may utilize a national database for identification verification at 256. It is then determined whether the identification is sufficient and passed at 258. If the identification was not sufficiently passed at 258, then the application collects updated identification information at 260. The updated information is then transferred back to the bank at 262 to approve the new information which the provides documents to the members at 264. Notably, if the identification was sufficient at 258, then the identification is passed and transfers directly to providing the documents to the member at 264. After the documents are provided to the member at 264, the forms should be signed at 266. Once the forms have been signed by the member at 266, the bank receives the documents at 268 and opens the account at 270. The account documents may be imaged at 272 and then the bank may transfer the new account files to the application at 274. After that, the files may be transferred from the new accounts from the bank to the application which is received and updates the member accounts at 276.

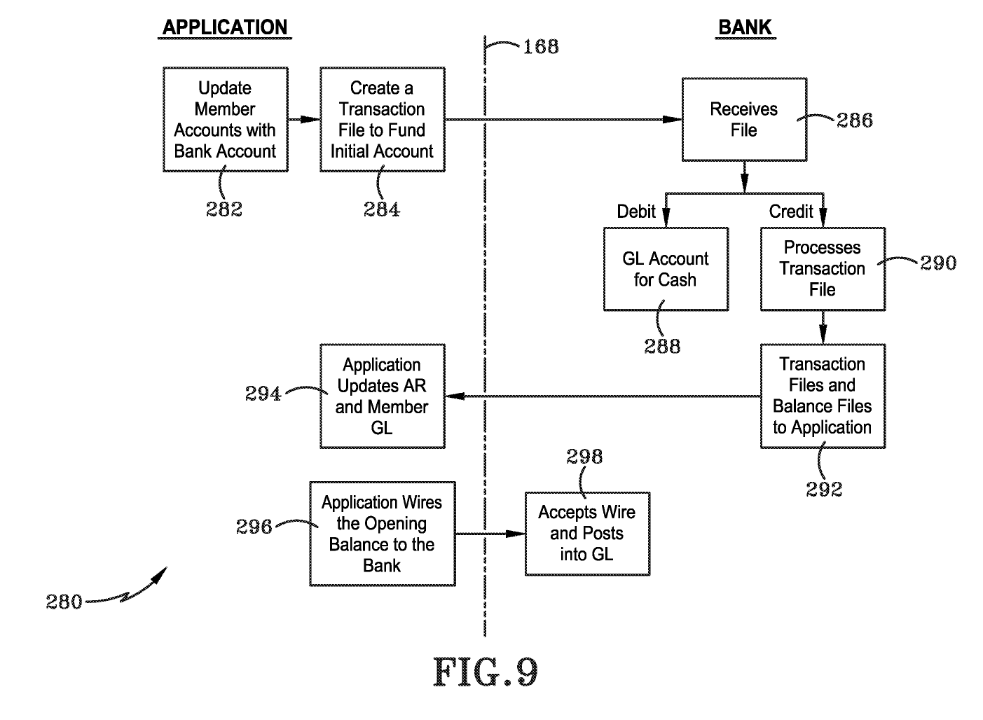

FIG. 9 depicts a conversion funding process generally at 280. The conversion funding process includes a similar imaginary line 168 representing a segregation between the application and the bank. With the conversion funding process 280, a member account may be updated with the bank account information in the application at 282. A transaction file to fund the initial account may be created in the application at 284. Then the information is transferred to the bank which receives the files at 286. After receiving the files at 286, the bank may debit the G/L account for cash at 288 and it may credit areas processes in the transaction file at 290. Subsequent to the credit of the process transaction files at 290, the transaction files and balance files may be provided to the application at 292 which are then received by the application and the application updates the accounts receivable and the member general ledger at 294. In the conversion funding process 280, the application may further wire the opening balance to the bank at 296 which is received by the bank which accepts the wire and posts the wire transfer into the general ledger account at 298.

In accordance with another aspect of the present disclosure, the system 10 and method for implementing the same provides improvements to another technological or technical field beyond that of normal computer processing. For example, banking information and cost sharing systems, such as MSAs are improved through the technological advancements provided herein. Additionally, some of the structural embodiments depicted herein provide improvements to the functioning of a computer itself, which is described in general detail below, which is utilized in conjunction with the servers and other diagrammatic embodiments. More particularly, the system 10 of the present disclosure enables and provides an improved functionality of a computer by enabling the cost sharing to occur directly between member-to-member accounts in a more efficient manner than has been previously provided. Through these improvements, the system 10 of the present disclosure adds a specific, meaningful, and novel limitation beyond what is well understood, routine, or conventional in the field. In other particular embodiments, the additional steps provided herein are unconventional inasmuch as they may narrow the system 10 of the present disclosure to a particular use. Additionally, the system 10 of the present disclosure, and the method for implementing the same, provide a solution to particular computer centric or Internet centric problems that were heretofore burdensome for conventional MSAs.

In accordance with non-limiting exemplary aspect of the present disclosure, some of the steps of the process or method may refer to a database stored within a computer or server depicted in FIG. 1. The database may include the information of members of the MSA of system 10. In these instances where the databases are utilized, the databases may be self-referential tables that improve the operation of the computer on which the system 10 is executed. In another particular example, the system 10 and methods for implementing the same may combine visual elements on a host website with content from a third-party merchant, such as the bank, to solve problems particular to experiences of direct member-to-member cost sharing scenarios. Collectively, this can be thought as a capability and improvement to the computer implemented system as a whole. Furthermore, the system 10 and its non-transitory computer readable storage mediums that have instructions or code stored thereon that, when executed by one or more processors, can be implemented with different types of processors without sacrificing any efficiency to the overall computer, thus improving the computer memory function.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of technology disclosed herein may be implemented using hardware, software, or a combination thereof. When implemented in software, the software code or instructions can be executed on any suitable processor or collection of processors, whether provided in a single computer or distributed among multiple computers. Furthermore, the instructions or software code can be stored in at least one non-transitory computer readable storage medium.

Also, a computer or smartphone utilized to executed the software code or instructions via its processors may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers or smartphones may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes outlined herein may be coded as software/instructions that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, USB flash drives, SD cards, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

The terms “program” or “software” or “instructions” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

All definitions, as defined and used herein, should be understood to control over dictionary definitions, definitions in documents incorporated by reference, and/or ordinary meanings of the defined terms.

“Logic”, as used herein, includes but is not limited to hardware, firmware, software and/or combinations of each to perform a function(s) or an action(s), and/or to cause a function or action from another logic, method, and/or system. For example, based on a desired application or needs, logic may include a software controlled microprocessor, discrete logic like a processor (e.g., microprocessor), an application specific integrated circuit (ASIC), a programmed logic device, a memory device containing instructions, an electric device having a memory, or the like. Logic may include one or more gates, combinations of gates, or other circuit components. Logic may also be fully embodied as software. Where multiple logics are described, it may be possible to incorporate the multiple logics into one physical logic. Similarly, where a single logic is described, it may be possible to distribute that single logic between multiple physical logics.

Furthermore, the logic(s) presented herein for accomplishing various methods of this system may be directed towards improvements in existing computer-centric or internet-centric technology that may not have previous analog versions. The logic(s) may provide specific functionality directly related to structure that addresses and resolves some problems identified herein. The logic(s) may also provide significantly more advantages to solve these problems by providing an exemplary inventive concept as specific logic structure and concordant functionality of the method and system. Furthermore, the logic(s) may also provide specific computer implemented rules that improve on existing technological processes. The logic(s) provided herein extends beyond merely gathering data, analyzing the information, and displaying the results. Further, portions or all of the present disclosure may rely on underlying equations that are derived from the specific arrangement of the equipment or components as recited herein. Thus, portions of the present disclosure as it relates to the specific arrangement of the components are not directed to abstract ideas. Furthermore, the present disclosure and the appended claims present teachings that involve more than performance of well-understood, routine, and conventional activities previously known to the industry. In some of the method or process of the present disclosure, which may incorporate some aspects of natural phenomenon, the process or method steps are additional features that are new and useful.

The term “rendered service” or “services rendered” as used herein refers to something that a person, company, etc., has done in exchange for a fee/payment. Examples have been provided herein which describe the provider as a doctor or medical facility and the rendered service as medical care provided by the doctor in exchange for a fee/payment thereof. However, the term “rendered service” may also refer to any service provided to the member of the MSA. For example, the provider that provides the rendered service may be in the field of, including by not limited to, accounting services, advertising services, audit services, auto services, building services, boat services, bridal services, business services, car rental agencies, catering services, children's services, check-cashing services, cleaning services, computer/IT services, consulting services, contractor services, copywriting and proof services, cover letter/resume services, dating services, decorating services, designing services, DJ businesses, dry cleaning and laundry services, dry cleaning delivery, editorial services, educational services, electrical services, employment services, environmental services, errand services, event planning, eye-care centers, financial services, fitness centers, golf services, hair salons, handyman services, health-care (allopathic, osteopathic, holistic, or other-types) services, home-improvement services, hospitality services, lawn care and landscaping, legal services, limousine services, locksmith services, maintenance services, marketing services, massage therapist, miscellaneous other services, moving services, painting services, personal-care services, personal chef, pest control services, pet-care services, photography services, plant maintenance services, plumbing services, pool services, postal and shipping/business services, printing services, private investigation, property inspection, property management services, publishing services, real estate services, recreational services, referral services, remodeling/renovation services, repair services, security services, self-defense instruction, senior care services, shipping services, shoe repair, sign stores, staffing services, tanning services, tax services, technology services, travel agencies, training businesses, translation services, tutoring services, videotaping services, web-site services, wedding services, or weight-control centers.

The indefinite articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the preferred embodiment of the disclosure are an example and the disclosure is not limited to the exact details shown or described.

1. A method for treating a patient by a medical provider and receiving payment for the treatment comprising:
treating, by a provider, a first patient who is a member of a mutual cost sharing association or organization (MSA) that does not collectively pool monies of MSA members that are provided to the MSA, wherein treating the first patient with a medical service defines a rendered service by the provider;
identifying, by the provider, the rendered service in a digital format;
transmitting the digital format of the rendered service from a provider computer to the MSA; and
receiving, by the provider, a payment from a bank in operative communication with the MSA for the rendered service to the first patient, wherein the payment includes monies from other MSA members having individual bank accounts at the bank.

2. The method for treating by the medical provider and receiving payment for the treatment of claim 1, wherein the payment from the bank to the provider does not include money from the first patient.

3. The method for treating by the medical provider and receiving payment for the treatment of claim 2, further comprising:
terminating, by the provider, an accounts receivable balance in response to receiving payment from the bank that includes money from other MSA members and not the first patient.

4. The method for treating by the medical provider and receiving payment for the treatment of claim 2, further comprising:
accessing, by the provider, a registry of at least one payment made by the bank; and
confirming, by the provider, the at least one payment was paid in full for the rendered service to the first patient.

5. The method for treating by the medical provider and receiving payment for the treatment of claim 2, further comprising:
agreeing, by the provider, to provider a second rendered service to the first patient in response to receiving payment from the bank, wherein the second render service is to be paid with monies from other MSA members that are not pooled prior to paying the provider.

6. The method for treating by the medical provider and receiving payment for the treatment of claim 1, further comprising:
processing, by the computer under the control of the provider, the payment defined by a plurality of monies form other MSA members distinct from the first patient.

7. The method for treating by the medical provider and receiving payment for the treatment of claim 1, further comprising:
aggregating, by the provider in a provider bank account, a plurality of monies received from other MSA members for the rendered service to the first patient, wherein the aggregating by the provider occurs after depositing the plurality of monies in the provider bank account such that no pooling of money occurs at the MSA or at the bank.

8. The method for treating by the medical provider and receiving payment for the treatment of claim 1, further comprising:
alerting, by the provider, the MSA in response to a notification from a provider bank that an insufficient amount of money was provided to the provider from the bank for the rendered service to the first patient.

9. The method for treating by the medical provider and receiving payment for the treatment of claim 1, further comprising:
associating, by the provider, the payment of monies from other MSA members to an account receivable of the first patient; and
confirming to the MSA that the account receivable of the first patient has been paid by the bank without the MSA pooling money of the MSA members.

10. The method for treating by the medical provider and receiving payment for the treatment of claim 1, further comprising:
allotting, by the provider, a number of line item entries at least equal to a number of payments received by the bank representing monies that are independent and retained in separate and independent bank accounts of other MSA members.

11. The method for treating by the medical provider and receiving payment for the treatment of claim 1, further comprising:
authenticating, by the provider, that the payment received from the bank has been paid from with monies from other MSA members that are independent and not pooled prior to paying the provider.

12. The method for treating by the medical provider and receiving payment for the treatment of claim 1, further comprising:
basing additional services rendered by the provider to the first member on payment of the rendered service with monies from other MSA members that are independent and not pooled prior to paying the provider.

13. The method for treating by the medical provider and receiving payment for the treatment of claim 1, further comprising:
billing, by the provider, the MSA to establish an account receivable at the provider and thereby establishing an expense for the first member at the MSA; and
notifying, by the provider, the MSA that the payment has been received with monies from other MSA members that remain independent prior to paying the provider so that the MSA notes that the expense for the first member has been paid.

14. The method for treating by the medical provider and receiving payment for the treatment of claim 1, further comprising:
bypassing the bank, by the provider, to notify the MSA that the payment has been received for the rendered service to the first member.

15. The method for treating by the medical provider and receiving payment for the treatment of claim 1, further comprising:
combining, by the provider, at a provider bank account monies received from the bank as payment for the rendered service, wherein monies are combined by the provider at the provider bank account and not before.

16. The method for treating by the medical provider and receiving payment for the treatment of claim 1, further comprising:
decreasing, by the provider, an account receivable entry associated with the rendered service to the first member in response to receiving payment from the bank for the rendered service with monies from other MSA members that are independent prior to paying the provider.

17. The method for treating by the medical provider and receiving payment for the treatment of claim 1, further comprising:
diagnosing, by the provider, the first member with a diagnosis as the rendered service prior to receiving payment from the bank for the rendered service with monies from other MSA members that are not pooled prior to paying the provider.

18. The method for treating by the medical provider and receiving payment for the treatment of claim 1, further comprising:
directing, by the provider, a provider bank account to receive receiving payment from the bank for the rendered service with monies from other MSA members that are not pooled prior to paying the provider is response to the MSA notifying the bank that a payment is to be made directly from the bank to the provider.

19. The method for treating by the medical provider and receiving payment for the treatment of claim 1, further comprising:
grouping, by the provider, at a provider bank account monies from other MSA members as the payment for the rendered service.

20. The method for treating by the medical provider and receiving payment for the treatment of claim 1, further comprising:
generating, by the provider, a notification sent to the MSA with instructions to be provided to the bank for the payment to be sent to a provider bank account.

21. The method for treating by the medical provider and receiving payment for the treatment of claim 1, further comprising:
instructing, by the provider, the MSA to initiate the payment from the bank with monies from other MSA members that are not pooled prior to paying the provider, wherein instructing the MSA is accomplished by sending an invoice for the rendered service.

22. The method for treating by the medical provider and receiving payment for the treatment of claim 1, further comprising:
initiating, by the provider, a second rendered service to the first member in response to the bank paying the provider the payment with monies from other MSA members.

23. The method for treating by the medical provider and receiving payment for the treatment of claim 1, further comprising:
limiting, by the provider, an additional number of services that can be rendered to the first member until the provider receives payment from the bank for the rendered service with monies from other MSA members that are not pooled prior to paying the provider.

24. The method for treating by the medical provider and receiving payment for the treatment of claim 1, further comprising:
maintaining, by the provider, a provider bank account; and
receiving in the provider bank account monies from other MSA members that are not pooled prior to paying the provider and are pooled by the bank, not the MSA, at the time the provider is paid.

25. The method for treating by the medical provider and receiving payment for the treatment of claim 1, further comprising:
minimizing, by the provider, additional services to be rendered to the first member until the provider has received payment from the bank for the rendered service with monies from other MSA members that are independent prior to paying the provider and are combined by the bank, not the MSA, at the time the provider is to be paid.

26. The method for treating by the medical provider and receiving payment for the treatment of claim 1, further comprising:
negotiating, by the provider, with the MSA a cost associated with the service, prior to rendering the service to the first member, wherein the cost associated with the service is based, at least partially, on a guarantee by the MSA that the bank will pay for the rendered service with monies from other MSA members that are not pooled prior to paying the provider and are pooled by the bank, not the MSA, at the time the provider is to be paid.

27. The method for treating by the medical provider and receiving payment for the treatment of claim 1, further comprising:
participating, by the provider, with the MSA to cooperate with the bank to receive payment for the rendered service with monies from other MSA members that remain independent prior to paying the provider and are pooled by the bank, not the MSA, at the time the provider is to be paid.

28. The method for treating by the medical provider and receiving payment for the treatment of claim 1, further comprising:
queuing, by the provider, an account receivable at a provider bank account for the rendered service to the first member with monies from other MSA members that remain independent prior to paying the provider and are combined by the bank at the time the provider is paid in one of a collective check and a direct funds transfer from the bank.

29. The method for treating by the medical provider and receiving payment for the treatment of claim 1, further comprising:
ranging, by the provider, a time to receive payment for the rendered service to the first member with monies from other MSA members that remain independent prior to paying the provider and are combined by the bank at the time the provider is paid in one of (i) a collective electronic or manual check drawn from the bank and (ii) a direct funds transfer from the bank.

30. The method for treating by the medical provider and receiving payment for the treatment of claim 1, further comprising:
registering, by the provider, that the payment from the bank has been received, wherein the payment is for the rendered service to the first member with monies from other MSA members that remain independent prior to paying the provider and are summed by the bank only at the time the provider is to be paid in the form of one of (i) a collective electronic or manual check drawn from the bank and (ii) a direct funds transfer from the bank.
